(19) United States
(12) Patent Application Publication
(10) Pub. No.: US 2013/0137990 A1
(43) Pub. Date: May 30, 2013

(54) ULTRASOUND ENDOSCOPE

(71) Applicant: OLYMPUS MEDICAL SYSTEMS CORP., Tokyo (JP)

(72) Inventor: Teppei TSURUTA, Tokyo (JP)

(73) Assignee: OLYMPUS MEDICAL SYSTEMS CORP., Tokyo (JP)

(21) Appl. No.: 13/681,862

(22) Filed: Nov. 20, 2012

Related U.S. Application Data
(63) Continuation of application No. PCT/JP2012/059794, filed on Apr. 10, 2012.

(30) Foreign Application Priority Data
May 20, 2011 (JP) 2011-113905

(51) Int. Cl. A61B 8/12

(52) U.S. Cl. CPC A61B 8/12
USPC 600/466

(57) ABSTRACT

An ultrasound endoscope includes an objective optical system, an illumination optical system, a distal end portion, an ultrasound transducer portion, a distal end cover, a through-hole formed in the distal end cover, a conductor wire portion, and a conductor wire portion passing portion formed in the distal end cover and configured to communicate with the through-hole and reach an outer circumference of the distal end cover, the conductor wire portion passing portion forming a region where the conductor wire portion passes from the through-hole to an outside of the distal end cover member when the ultrasound transducer portion is detached from the distal end portion.

### CROSS REFERENCE TO RELATED APPLICATION

This application is a continuation application of PCT/JP2012/059794 filed on Apr. 10, 2012 and claims benefit of Japanese Application No. 2011-113905 filed in Japan on May 20, 2011, the entire contents of which are incorporated herein by this reference.

### BACKGROUND OF THE INVENTION

1. Field of the Invention

The present invention relates to an ultrasound endoscope, a distal end portion of an insertion portion of which is covered by a distal end cover.

2. Description of the Related Art

In an ultrasound endoscope that can observe an ultrasound image, which is a two-dimensional visible image of a region to be examined, a configuration is well known in which a distal end cover is provided in an outer circumference of a distal end portion made of metal located on a distal end side in an inserting direction of an insertion portion. The configuration is disclosed in, for example, Japanese Patent Application Laid-Open Publication No. 2005-323886.

The distal end cover is made of an insulating member. The distal end cover has a function of covering the outer circumference of the distal end portion to thereby protect the distal end portion besides retaining insulation properties of the distal end portion.

Note that, on a distal end face, for example, distal end openings for an objective optical system, an illumination optical system, and a treatment instrument insertion channel and an opening of a through-hole are provided. A proximal end portion in an inserting direction of an ultrasound transducer portion provided at the distal end is attached to the opening of the through-hole such that at least a region including a piezoelectric element projects to a front in the inserting direction from the distal end face of the distal end portion.

A configuration is naturally conceivable in which a region on the distal end face excluding the openings for the objective optical system, the illumination optical system, and the treatment instrument insertion channel, the opening of the through-hole to which the proximal end portion of the ultrasound transducer portion is attached, and the like is covered using the distal end cover.

Usually, an image pickup unit including the objective optical system, an illumination unit including the illumination optical system, and the like are provided in the distal end portion by being fixed to the distal end portion by screws or the like. If these members arranged in the distal end portion are replaced, when the screws are removed, it is sometimes necessary to detach the distal end cover from the distal end portion in order to expose the screws.

In the configuration in which the distal end cover covers the distal end face of the distal end portion, a distal end side region in the inserting direction of the ultrasound transducer portion is formed larger in an outer diameter according to a shape of the piezoelectric element than a hole formed in the distal end cover that covers the opening to which the proximal end side of the ultrasound transducer portion is attached. Therefore, it is necessary to first detach the ultrasound transducer portion from the distal end portion, and then detach the distal end cover from the distal end portion.

Note that a distal end in an inserting direction of a conductor wire portion for performing at least exchange of electric power and an electric signal with the ultrasound transducer portion is electrically connected to the ultrasound transducer portion. The conductor wire portion is inserted through the insertion portion of the ultrasound endoscope, an operation portion provided at a proximal end in the inserting direction of the insertion portion, a universal cord extending from the operation portion, and a connector provided at an extension end of the universal cord. The conductor wire portion has a predetermined length.

Therefore, when the ultrasound transducer portion is detached from the distal end portion, it is necessary to disconnect a proximal end region in the inserting direction of the conductor wire portion located in the operation portion and in the connector, and then draw out, together with the ultrasound transducer portion, the conductor wire portion further forward in the inserting direction than the distal end face of the distal end portion.

After drawing out the conductive wire portion, in order to detach the distal end cover from the conductor wire portion, there is no choice but to draw out the entire conductor wire portion having predetermined length to the front in the inserting direction via the hole of the distal end cover because a diameter of the hole that covers the opening in which the proximal end portion of the ultrasound transducer portion of the distal end cover is fit is smaller than the outer diameter on the ultrasound transducer portion distal end side.

### SUMMARY OF THE INVENTION

An ultrasound endoscope according to an aspect of the present invention includes: an objective optical system; an illumination optical system configured to illuminate a visual field of the objective optical system; a distal end portion located on a distal end side in an inserting direction of an insertion portion inserted in to a subject, at least the objective optical system and the illumination optical system being fixed to the distal end portion; an ultrasound transducer portion detachably attachable to the distal end portion, at least a region of which including a piezoelectric element projecting further forward in the inserting direction than a distal end face of the distal end portion; a distal end cover detachably attachable to the distal end portion, the distal end cover covering an outer circumferential surface and the distal end face of the distal end portion such that at least the objective optical system, the illumination optical system, and a region including the piezoelectric element of the ultrasound transducer portion is exposed from the distal end face of the distal end portion; a through-hole formed in the distal end cover to pierce through the distal end cover along the inserting direction and closed by a part of the ultrasound transducer portion when the ultrasound transducer portion is attached to the distal end portion; a conductor wire portion, one end of which is electrically connected to the piezoelectric element, the conductor wire portion piercing through the distal end portion in the inserting direction and piercing through the through-hole when the ultrasound transducer portion is detached from the distal end portion; and a conductor wire portion passing portion formed in the distal end cover and configured to communicate with the through-hole and reach an outer circumference of the distal end cover, the conductor wire portion passing portion forming a region where the conductor wire portion passes from the through-hole to an outside of the distal end cover member when the ultrasound transducer portion is detached from the distal end portion.

### BRIEF DESCRIPTION OF THE DRAWINGS

FIG. 1 is an exploded perspective view of a distal end portion in an insertion portion of an ultrasound endoscope according to a first embodiment;

FIG. 2 is a diagram of the distal end portion shown in FIG. 1 schematically viewed from a II direction in FIG. 1 together with a bending portion;

FIG. 3 is a diagram showing a state in which a distal end cover, an air/water feeding nozzle, and an ultrasound transducer unit are attached to a distal end rigid member of the distal end portion shown in FIG. 2;

FIG. 4 is a diagram of the distal end portion shown in FIG. 3 viewed from a IV direction in FIG. 3;

FIG. 5 is a sectional view of the distal end portion taken along a V-V line in FIG. 3;

FIG. 6 is a diagram schematically showing a distal end side of an insertion portion of an ultrasound endoscope according to a second embodiment;

FIG. 7 is a sectional view of a distal end portion taken along a VII-VII line in FIG. 6;

FIG. 8 is an exploded view schematically showing a distal end side of an insertion portion of an ultrasound endoscope according to a third embodiment;

FIG. 9 is a sectional view of an ultrasound transducer unit taken along a IX-IX line in FIG. 8;

FIG. 10 is a sectional view of a distal end portion taken along a X-X line in FIG. 8; and

FIG. 11 is a diagram showing an external appearance of the ultrasound endoscope in which the ultrasound transducer unit is provided in FIG. 1.

### DETAILED DESCRIPTION OF THE PREFERRED EMBODIMENTS

Embodiments of the present invention are explained below with reference to the drawings. It should be noted that the drawings are schematic and relations among thicknesses and widths of respective members, ratios of the thicknesses of the respective members, and the like are different from real ones. It goes without saying that portions where relations and ratios of dimensions are different from one another are included among the drawings.

### First Embodiment

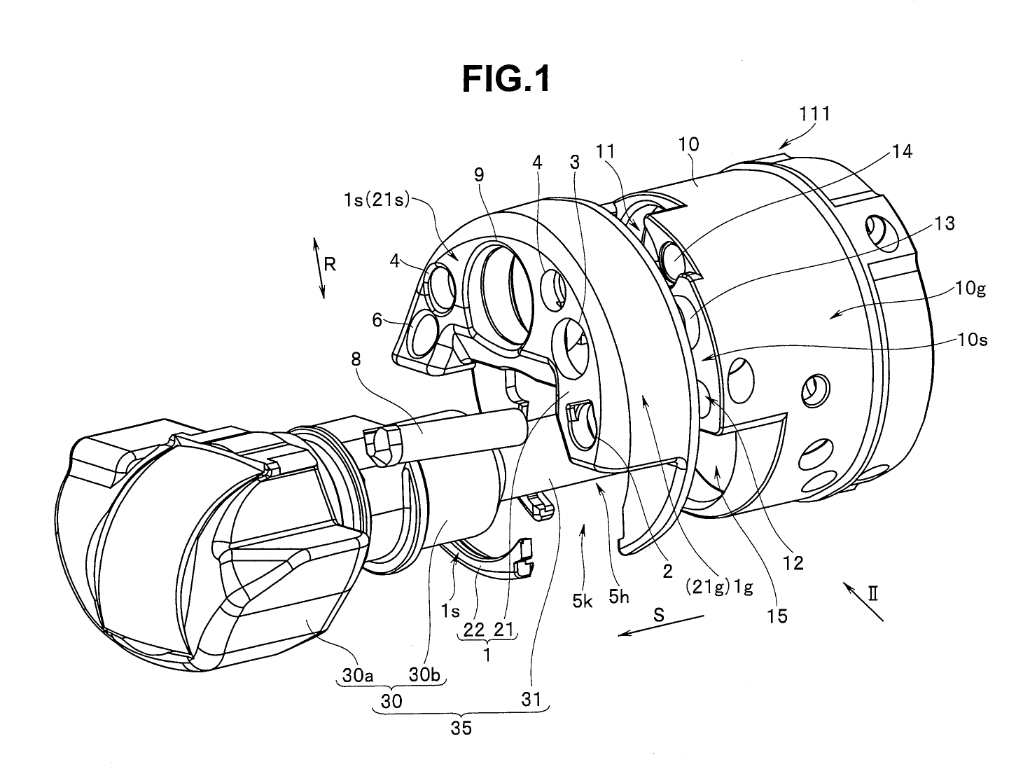

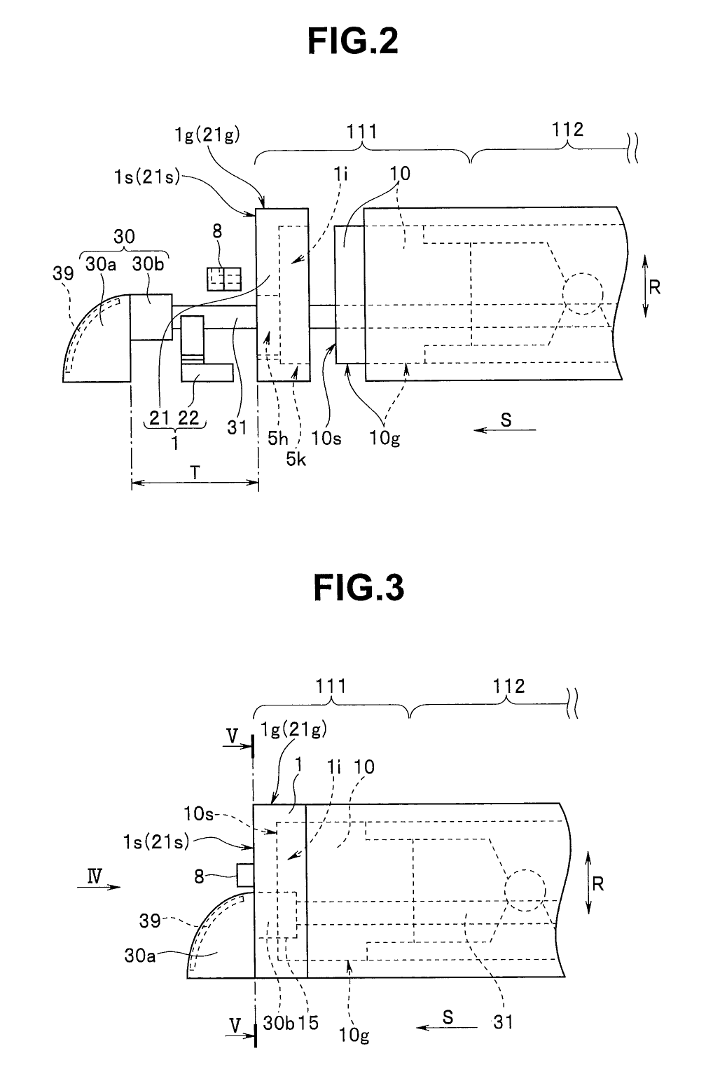

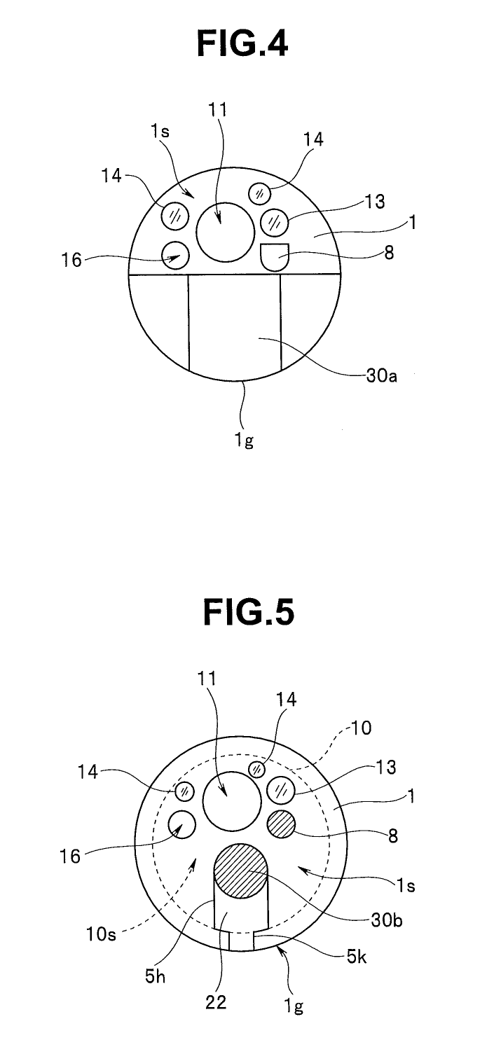

FIG. 1 is an exploded perspective view of a distal end portion in an insertion portion of an ultrasound endoscope according to a first embodiment. FIG. 2 is a diagram of the distal end portion shown in FIG. 1 schematically viewed from a II direction in FIG. 1 together with a bending portion. FIG. 3 is a diagram showing a state in which a distal end cover, an air/water feeding nozzle, and an ultrasound transducer unit are attached to a distal end rigid member of the distal end portion shown in FIG. 2. FIG. 4 is a diagram of the distal end portion shown in FIG. 3 viewed from a IV direction in FIG. 3. FIG. 5 is a sectional view of the distal end portion taken along a V-V line in FIG. 3.

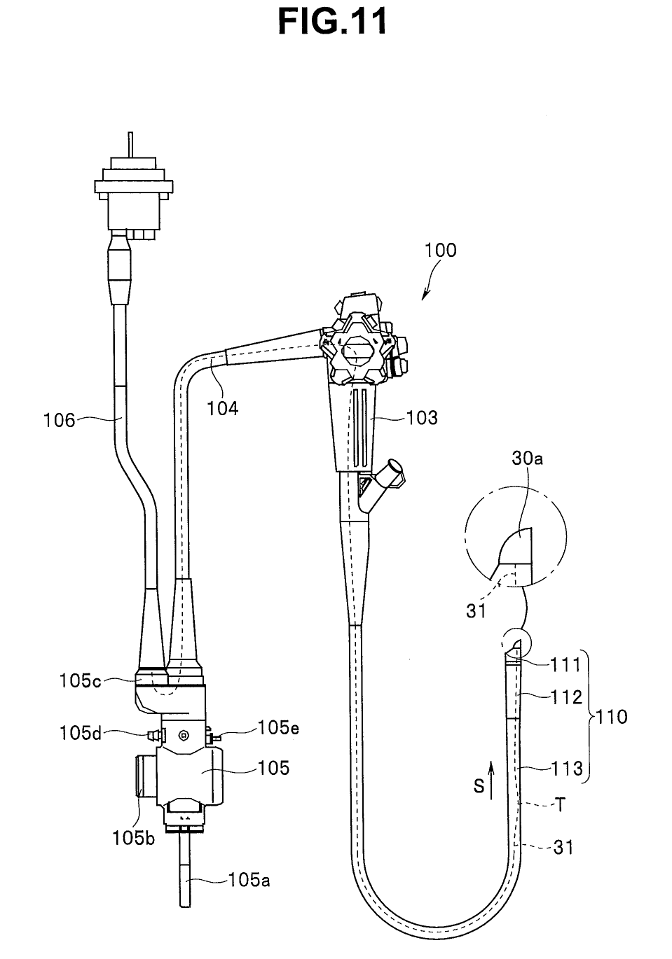

As shown in FIGS. 1 to 3, a distal end portion 111 located at a distal end of an inserting direction S in an insertion portion 110, which is inserted into a subject, of an ultrasound endoscope 100 explained below (for the insertion portion 110 and the ultrasound endoscope 100, see FIG. 11) includes a distal end rigid member 10. The distal end rigid member 10 is made of, for example, metal.

In the distal end rigid member 10, a distal end side in the inserting direction S of an ultrasound transducer unit 35, a distal end side in the inserting direction S of a treatment instrument insertion channel 11, an image pickup unit and an illumination unit, a distal end side in the inserting direction of an air/water feeding conduit 12, a distal end side in the inserting direction of a forward water feeding conduit 16 (see FIGS. 4 and 5), and the like are provided along the inserting direction S.

Note that the treatment instrument insertion channel 11, the air/water feeding conduit 12, and the forward water feeding conduit 16 are inserted through an operation portion 103, a universal cord 104, and a connector 105 (for all of which, see FIG. 11) besides the insertion portion 110 of the ultrasound endoscope 100.

A main portion of the ultrasound transducer unit 35 includes an ultrasound transducer portion 30 including a first region 30a and a second region 30b and a conductor wire portion 31, one end of which is electrically connected to the ultrasound transducer portion 30. The conductor wire portion 31 performs at least exchange of electric power and an electric signal with the ultrasound transducer portion 30.

The conductor wire portion 31 is inserted through the connector 105, the universal cord 104, the operation portion 103, and the insertion portion 110 of the ultrasound endoscope 100, explained below (for all of which, see FIG. 11). Note that the conductor wire portion 31 is inserted through the insertion portion 110 in a state in which the conductor wire portion 31 is loosened by a predetermined length T, for example, about several centimeters in the insertion portion 110.

The conductor wire portion 31 usually includes a configuration in which a knitted shield formed by knitting metal element wires for improving anti-noise properties is coated on an outer circumference of a plurality of twisted coaxial lines, in which signal lines, GND lines, insulating materials, and skins are coaxially provided, and a skin of resin or the like is coated on an outer circumference of the knitted shield.

However, in the configuration in which the knitted shield is used, although the anti-noise properties are high, there is a drawback in that the knitted shield that expands and contracts or bends following bending and tension applied to the insertion portion 110 tends to be broken.

Therefore, in the present embodiment, a configuration is adopted in which a tape of copper foil or the like for reducing radiation noise from the coaxial lines is spirally wound around an outer circumference from a distal end to a proximal end in the inserting direction of the plurality of twisted coaxial lines and a wire element for shield is wound around an outer circumference from a distal end to a proximal end in the inserting direction of the tape.

With such a configuration, it is possible to maintain anti-noise properties equivalent to the knitted shield. Further, since the copper foil tape has high flexibility, the copper foil tape is capable of extending and contracting. Therefore, it is possible to improve mechanical strength.

This is for preventing a hardened portion from being partially formed in a predetermined place of the knitted shield by friction that occurs among the metal element wires according to the expansion and contraction or the bending of the knitted shield.

When the signal lines of the plurality of coaxial lines in the conductor wire portion 31 are electrically connected to a substrate of the ultrasound transducer unit 35, if the knitted shield is coated, there is a drawback in that it is difficult to expose ends of the signal lines. However, with the present configuration, ends of the copper tape and the element wires are easily cut. Therefore, it is possible to easily expose the ends of the signal lines.

The element wires wound around the copper foil tape can be formed thinner in a radial direction R than the knitted shield. Therefore, it is possible to reduce a diameter of the insertion portion 110.

Note that the configuration explained above can be applied not only to the conductor wire portion 31 that performs at least exchange of electric power and an electric signal with the ultrasound transducer portion 30 but also to the signal lines extended from the image pickup unit.

The ultrasound transducer unit 35 is detachably attachable to the distal end rigid member 10. The ultrasound transducer unit 35 is provided such that, after being attached, a distal end in the inserting direction S projects further forward in the inserting direction S than a distal end face 10s of the distal end rigid member 10.

Specifically, as shown in FIGS. 3 and 5, in the ultrasound transducer portion 30 provided on the distal end side in the inserting direction S of the ultrasound transducer unit 35, the first region 30a is provided to project further forward in the inserting direction S than the distal end face 10s of the distal end rigid member 10.

Note that, on an inside of the first region 30a covered with a housing, an ultrasound transducer is provided. The ultrasound transducer includes a piezoelectric element 39 having a function of radiating, according to vibration, ultrasound on a region to be examined and receiving a reflected sound wave from the region to be examined and converting the reflected sound wave into an electric signal, a GND electrode and a signal electrode that apply a voltage to the piezoelectric element 39, a lens that collects the ultrasound, an acoustic matching layer, a backing material, a substrate to which the signal electrode is electrically connected and the like.

On an inside of the second region 30b covered with a housing, one ends of the signal lines of the conductor wire portion 31 are electrically connected to the substrate of the ultrasound transducer. In the ultrasound transducer portion 30, the conductor wire portion 31 is electrically connected to the piezoelectric element 39 via the substrate and the signal electrode.

In the distal end rigid member 10, a hole 15 that pierces through the distal end rigid member 10 along the inserting direction S is provided. A proximal end side in the inserting direction S of the second region 30b of the ultrasound transducer portion 30 can be fit in the hole 15. The distal end side in the inserting direction S of the conductor wire portion 31 is pierced through the hole 15.

Note that, the proximal end side in the inserting direction S of the second region 30b is fit in the hole 15 and fixed by screws or the like, whereby the ultrasound transducer unit 35 is attached to the distal end rigid member 10. In other words, the ultrasound transducer unit 35 is detachably attachable to the distal end rigid member 10 by the screws or the like.

The distal end side in the inserting direction S of the treatment instrument insertion channel 11, the image pickup unit and the illumination unit, the distal end side in the inserting direction of the air/water feeding conduit 12, the distal end side in the inserting direction of the forward water feeding conduit 16, and the like are also detachably attachable to the distal end rigid member 10 by screws or the like.

Further, as shown in FIG. 5, on the distal end face 10s of the distal end rigid member 10, a distal end opening of the hole 15, an objective optical system 13 included in the image pickup unit, an illumination optical system 14 included in the illumination unit and configured to illuminate a visual field of the objective optical system 13, a distal end opening of the treatment instrument insertion channel 11, a distal end opening of the air/water feeding conduit 12, a distal end opening of the forward water feeding conduit 16, and the like are provided.

Note that, as shown in FIG. 1, the distal end face 10s has a step shape. The distal end opening of the hole 15 is formed in a position of the distal end face 10s recessed further backward in the inserting direction S than a position where the objective optical system 13 and the distal end opening of the air/water feeding conduit 12 are provided such that, when the proximal end side of the second region 30b of the ultrasound transducer portion 30 is attached to the hole 15, the first region 30a projects further forward in the inserting direction S than the distal end face 10s.

On the distal end face 10s, the illumination optical system 14 and the distal end opening of the forward water feeding conduit 16 are also formed in a position recessed further backward in the inserting direction than the position where the objective optical system 13 and the distal end opening of the air/water feeding conduit 12 are provided.

Further, on the distal end face 10s, the distal end opening of the treatment instrument insertion channel 11 is also formed in a position recessed further backward in the inserting direction than a position where the illumination optical system 14 and the distal end opening of the forward water feeding conduit 16 are provided.

Note that an air/water feeding nozzle 8 for feeding fluid to the objective optical system 13 is detachably attachable to the distal end opening of the air/water feeding conduit 12. After the air/water feeding nozzle 8 is attached to the distal end opening of the air/water feeding conduit 12, as shown in FIGS. 3 and 5, the distal end side in the inserting direction S of the air/water feeding nozzle 8 is located to project further forward in the inserting direction S than the distal end face 10s.

As shown in FIGS. 1 to 4, a distal end cover 1 that covers an outer circumferential surface 10g and the distal end face 10s of the distal end rigid member 10 is detachably attachable to the distal end rigid member 10 via an adhesive or the like. In other words, the distal end rigid member 10 can be fit in an inside 1i of the distal end cover 1.

Note that the distal end cover 1 is made of resin having insulation properties and chemical resistance, for example, polyphenyl sulfone, polysulfone, or PEEK. A main portion of the distal end cover 1 includes a first distal end cover portion 21 and a second distal end cover portion 22.

As shown in FIGS. 1 to 3, the main portion of the distal end cover 1 includes a distal end face 1s serving as a region that covers the distal end face 10s and an outer circumferential surface 1g serving as a region that covers the outer circumferential surface 10g after the distal end cover 1 is attached to the distal end rigid member 10.

On a distal end face 21s of the first distal end cover portion 21 forming the distal end face 1s, as shown in FIG. 1, through-holes 5h, 3, 4, 9, 2, and 6 along the inserting direction S are respectively formed such that the first region 30a of the ultrasound transducer portion 30, the objective optical system 13, the illumination optical system 14, the distal end opening of the treatment instrument insertion channel 11, the air/water feeding nozzle 8, and the distal end opening of the forward water feeding conduit 16 are exposed from the distal end face 10s of the distal end rigid member 10.

The through-hole 5h exposes the first region 30a of the ultrasound transducer portion 30 from the distal end face 10s. After the proximal end side of the second region 30b of the ultrasound transducer portion 30 is attached to the hole 15 from the front in the inserting direction S via the through-hole 5h, the through-hole 5h is closed by a region on the distal end side of the second region 30b. The through-hole 5h forms a region through which the conductor wire portion 31 pierces when the ultrasound transducer unit 35 is detached from the distal end rigid member 10.

Note that, as explained above, the conductor wire portion 31 is inserted through the insertion portion 110 in the state in which the conductor wire portion 31 is loosened by a predetermined length T, for example, several centimeters in the insertion portion 110. Therefore, when the ultrasound transducer unit 35 is detached from the distal end rigid member 10, the ultrasound transducer portion 30 can project further forward in the inserting direction S than the distal end face 10s of the distal end rigid member 10 by the length of the loosening of the conductor wire portion 31, for example, about several centimeters.

The through-hole 3 exposes the objective optical system 13 from the distal end face 10s. The through-hole 4 exposes the illumination optical system 14 from the distal end face 10s. The through-hole 9 exposes the distal end opening of the treatment instrument insertion channel 11 from the distal end face 10s. The through-hole 2 exposes the air/water feeding nozzle 8 from the distal end face 10s. The through-hole 6 exposes the distal end opening of the forward water feeding conduit 16 from the distal end face 10s.

Note that, after being attached to the distal end rigid member 10, the distal end cover 1 is pressed against and bonded to the distal end face 10s by the air/water feeding nozzle 8 and the first region 30a of the ultrasound transducer portion 30.

As shown in FIGS. 1 and 2, in the first distal end cover portion 21, a cutout portion 5k is formed. The cutout portion 5k is a conductor wire passing portion that causes the through-hole 5h and an outer circumferential surface 21g of the first distal end cover portion 21, which forms the outer circumferential surface 1g of the distal end cover 1, to communicate with each other along a radial direction R of the distal end rigid member 10. In other words, the first distal end cover portion 21 has a shape which is cut out in the radial direction R by the cutout portion 5k to communicate the through-hole 5h with the outside of the distal end cover 1.

The cutout portion 5k forms a region where the conductor wire portion 31 passes in the radial direction R from the through-hole 5h to the outside of the distal end cover 1 in order to detach the first distal end cover portion 21 from the ultrasound transducer unit 35 when the ultrasound transducer unit 35 is detached from the distal end rigid member 10, specifically, when the ultrasound transducer portion 30 is drawn out to the front in the inserting direction S by the length of the loosening of the conductor wire portion 31 from the distal end face 10s via the through-hole 5h.

The second distal end cover portion 22 is detachably attachable to the cutout portion 5k of the first distal end cover portion 21 from, for example, the front in the inserting direction S. After being attached, as shown in FIGS. 3 and 5, the second distal end cover portion 22 closes the cutout portion 5k. Note that, although not shown in the figure, the second distal end cover portion 22 includes a slip-off preventing mechanism for preventing the second distal end cover portion 22 from suddenly coming off the cutout portion 5k after being attached.

Next, action of the present embodiment is explained.

When, for example, the image pickup unit provided in the distal end rigid member 10 breaks down and needs to be replaced, firstly, an operator detaches, by releasing the screws or the like, the ultrasound transducer portion 30 fixed to the hole 15 of the distal end rigid member 10. As shown in FIG. 1, the operator draws out the ultrasound transducer portion 30 to the front in the inserting direction S by the length of the loosening of the conductor wire portion 31 in the insertion portion 110. When the ultrasound transducer portion 30 is drawn out, the conductor wire portion 31 passes through the through-hole 5h of the distal end cover 1.

Thereafter, as shown in FIG. 1, the operator detaches the air/water feeding nozzle 8 from the distal end opening of the air/water feeding conduit 12 by drawing out the air/water feeding nozzle 8 to the front in the inserting direction S. Note that, after the ultrasound transducer portion 30 is drawn out and the air/water feeding nozzle 8 is detached, nothing presses the distal end cover 1 against the distal end face 10s of the distal end rigid member 10 to a back in the inserting direction S.

Subsequently, the operator detaches, in the distal end cover 1, the second distal end cover portion 22 from the cutout portion 5k of the first distal end cover portion 21.

Thereafter, the operator releases the bonding of the first distal end cover portion 21 to the distal end rigid member 10 and draws out the first distal end cover portion 21 from the distal end rigid member 10 to the front in the inserting direction S.

Subsequently, the operator moves the conductor wire portion 31 in the radial direction R and removes the conductor wire portion 31 from the through-hole 5h to the outside of the distal end cover 1 via the cutout portion 5k, whereby the distal end cover 1 is detached from the ultrasound transducer unit 35.

When the distal end cover 1 is detached, since the cutout portion 5k is formed in the distal end cover 1, it is possible to easily detach the distal end cover 1 from the conductor wire portion 31 in the radial direction R via the cutout portion 5k without drawing out the entire conductor wire portion 31 to the front in the inserting direction S via the through-hole 5h unlike in the past. Note that, usually, the distal end cover 1 is discarded after being detached. However, the distal end cover 1 may be reused.

Thereafter, the image pickup unit is detached from the distal end rigid member 10 via the screws or the like, replaced or repaired, and attached to the distal end rigid member 10 again.

Subsequently, the operator attaches a new distal end cover 1 via the cutout portion 5k by leading the conductor wire portion 31 into the through-hole 5h of the first distal end cover portion 21 from an outer side in the radial direction R. When the new distal end cover 1 is attached, since the cutout portion 5k is formed, it is possible to easily insert the conductor wire portion 31 through the through-hole 5h of the new distal end cover 1.

Thereafter, in order to prevent the conductor wire portion 31 from coming off the through-hole 5h, the operator closes the cutout portion 5k of the first distal end cover portion 21 with the second distal end cover portion 22 and places the distal end cover 1 over the distal end face 10s and the outer circumferential surface 10g of the distal end rigid member 10 from the front in the inserting direction S and fixes the distal end cover 1 with an adhesive.

Finally, the operator pushes in the second region 30b of the ultrasound transducer portion 30 from the front in the inserting direction S to the back in the inserting direction S, fits the proximal end side of the second region 30b in the hole 15 via the through-hole 5h, and thereafter fixes the proximal end side of the second region 30b with screws or the like.

In this way, the image pickup unit is replaced. The above explanation is not limited to the replacement of the image pickup unit. The same holds true in replacement work for various members provided in the distal end rigid member 10 and also holds true in replacement work for the distal end cover 1 itself.

As explained above, in the present embodiment, the distal end face 10s of the distal end rigid member 10 is also covered by the distal end cover 1.

Consequently, it is possible to prevent, using the distal end cover 1 made of a member having insulation properties, a situation in which, when a high-frequency cauterization treatment instrument is projected to the front in the inserting direction S to perform cauterization treatment of a region to be examined, the high-frequency cauterization treatment instrument comes into contact with the distal end face 10s of the distal end rigid member 10, which is made of metal, directly or via conductive liquid such as water containing impurities, and the distal end face 10s is cauterized. Further, it is possible to prevent, using the distal end cover 1 having chemical resistance, a situation in which a chemical adheres to the distal end face 10s and the distal end face 10s is chemically damaged.

In the present embodiment, the cutout portion 5k of the through-hole 5h is formed in the first distal end cover portion 21 of the distal end cover 1. The cutout portion 5k is closed by the second distal end cover portion 22.

Consequently, when the distal end cover 1 is detached from the distal end rigid member 10, it is possible to easily detach the distal end cover 1 simply by, after drawing out the ultrasound transducer portion 30 of the ultrasound transducer unit 35 to the front in the inserting direction S by the length of the loosening of the conductor wire portion 31 in the insertion portion 110, drawing out the distal end cover 1 from the distal end rigid member 10 to the front in the inserting direction S, thereafter detaching the second distal end cover portion 22 from the cutout portion 5k, and allowing the conductor wire portion 31 inserted through the through-hole 5h of the distal end cover 1 to pass to the outside of the distal end cover 1 via the cutout portion 5k.

When the new distal end cover 1 is attached, it is possible to easily attach the distal end cover 1 simply by leading the conductor wire portion 31 into the through-hole 5h via the cutout portion 5k and, thereafter, after closing the cutout portion 5k with the second distal end cover portion 22, attaching the distal end cover 1 and the ultrasound transducer portion 30 to the distal end rigid member 10.

Therefore, unlike the conventional cases, it is unnecessary to draw out the entire conductor wire portion 31 from the through-hole 5h of the distal end cover 1 from the ultrasound endoscope 100 to the front in the inserting direction S and lead the entire conductor wire portion 31 into the ultrasound endoscope 100 again via the through-hole 5h.

Consequently, it is possible to provide the ultrasound endoscope 100 including the configuration in which the distal end cover 1 that covers the distal end face 10s together with the outer circumferential surface 10g of the distal end rigid member 10 of the distal end portion 111 can be easily attached and detached.

### Second Embodiment

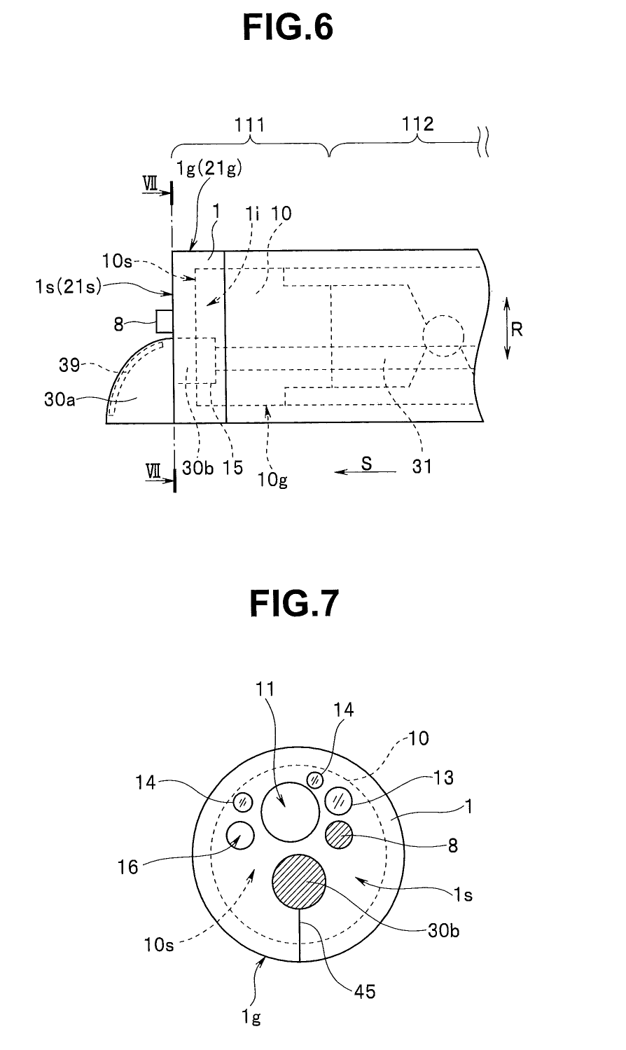

FIG. 6 is a diagram schematically showing a distal end side of an insertion portion of an ultrasound endoscope according to a second embodiment. FIG. 7 is a sectional view of a distal end portion taken along a VII-VII line in FIG. 6.

A configuration of the ultrasound endoscope according to the second embodiment is different from the ultrasound endoscope according to the first embodiment shown in FIGS. 1 to 5 in that a distal end cover is formed by one member and a conductor wire passing portion provided in the distal end cover is formed by a slit. Therefore, only the difference is explained. Components same as the components in the first embodiment is denoted by the same reference numerals and signs and explanation of the components is omitted.

As shown in FIGS. 6 and 7, in the present embodiment, the distal end cover 1 is formed by one member.

In the distal end cover 1, in order to detach the distal end cover 1 from the ultrasound transducer unit 35 when the ultrasound transducer portion 30 is drawn out to the front in the inserting direction S by the length of the loosening of the conductor wire portion 31 from the distal end face 10s via the through-hole 5h, the conductor wire passing portion forming the region where the conductor wire portion 31 passes from the through-hole 5h to the outside of the distal end cover 1 is formed by a linear slit 45 formed in the distal end cover 1.

Note that the slit 45 causes the through-hole 5h and the outer circumferential surface 1g of the distal end cover 1 to communicate with each other in the radial direction R. Slit width of the slit 45 is set smaller than a diameter of the conductor wire portion 31.

The slit 45 is a region where the conductor wire portion 31 passes when the conductor wire portion 31 is removed from the through-hole 5h to the outside of the distal end cover 1 or when the conductor wire portion 31 is led into the through-hole 5h from the outside of the distal end cover 1. When the conductor wire portion 31 is removed or when the conductor wire portion 31 is led in, an operator twists and deforms a vicinity of the slit 45 of the distal end cover 1 and increases slit width of the slit 45 to thereby allow the conductor wire portion 31 to pass.

Therefore, the distal end cover 1 in the present embodiment is desirably formed by a member softer than the member forming the distal end cover 1 in the first embodiment. Other components and actions are the same as the components and the actions in the first embodiment explained above.

With such a configuration, since the distal end cover 1 can be formed by one member, it is possible to further reduce manufacturing costs than in the first embodiment. Further, since work for detaching the second distal end cover portion 22 is unnecessary, it is possible to improve workability. Other effects are the same as the effects in the first embodiment explained above.

### Third Embodiment

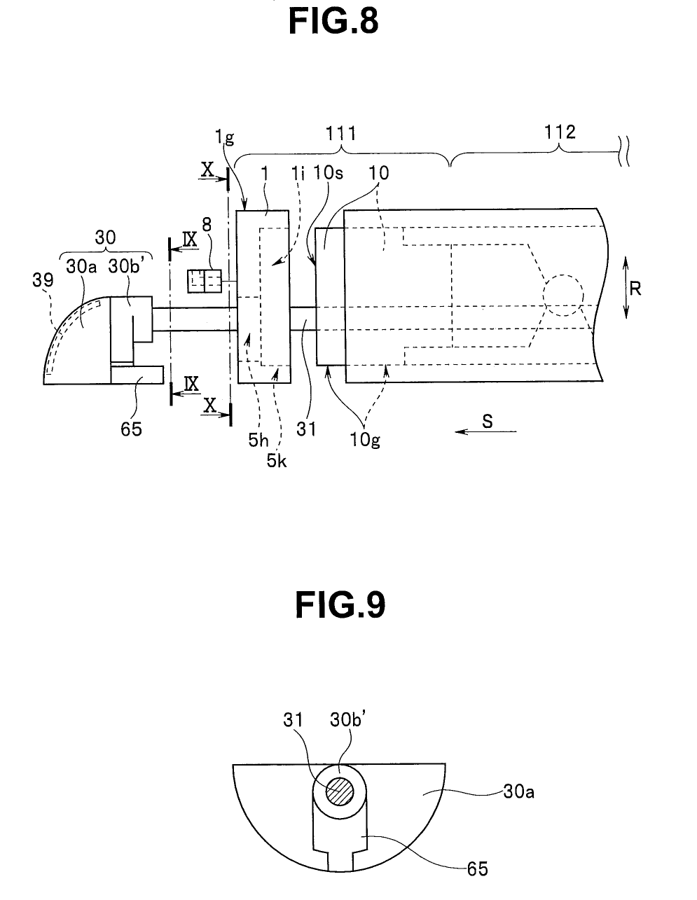

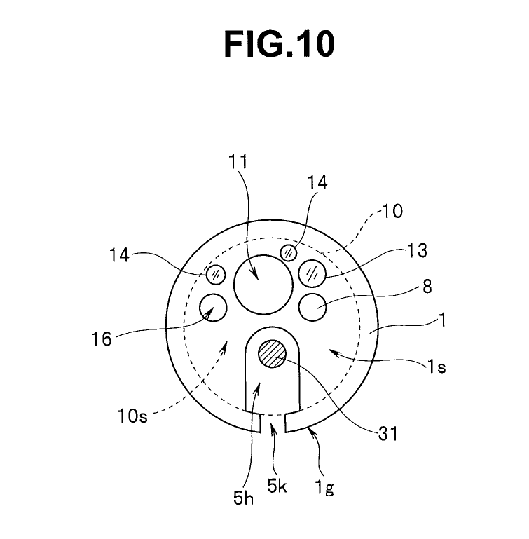

FIG. 8 is an exploded view schematically showing a distal end side of an insertion portion of an ultrasound endoscope according to a third embodiment. FIG. 9 is a sectional view of an ultrasound transducer unit taken along a IX-IX line in FIG. 8. FIG. 10 is a sectional view of a distal end portion taken along a X-X line in FIG. 8.

A configuration of the ultrasound endoscope according to the third embodiment is different from the ultrasound endoscope according to the first embodiment shown in FIG. 1 through FIG. 5 in that a member that closes a cutout portion of a distal end cover is provided in an ultrasound transducer portion. Therefore, only the difference is explained. Components same as the components in the first embodiment is denoted by the same reference numerals and signs and explanation of the components is omitted.

As shown in FIGS. 8 and 10, in the present embodiment, as in the first embodiment, in the distal end cover 1, the cutout portion 5k that causes the through-hole 5h and the outer circumferential surface 1g to communicate with each other in the radial direction R is formed. Note that action of the cutout portion 5k in the present embodiment is the same as the action in the first embodiment. Therefore, explanation of the action is omitted.

As shown in FIGS. 8 and 9, in a housing of a second region 30b′ of the ultrasound transducer portion 30, a closing member 65 that closes the cutout portion 5k when a proximal end side of the second region 30b′ of the ultrasound transducer portion 30 is fit in the hole 15 of the distal end rigid member 10 is provided.

Note that the closing member 65 may be formed integrally with the housing of the second region 30b′ or may be provided separately from the housing of the second region 30b′.

With such a configuration, as in the first embodiment, the cutout portion 5k is formed simply by drawing out the ultrasound transducer portion 30 from the distal end rigid member 10 to the front in the inserting direction S. Therefore, after the distal end cover 1 is drawn out to the front in the inserting direction S, as in the first embodiment, it is possible to easily remove the conductor wire portion 31 from or lead the conductor wire portion 31 into the through-hole 5h via the cutout portion 5k.

Therefore, even if the distal end cover 1 is formed by one member, it is possible to surely close the cutout portion 5k with the closing member 65 of the ultrasound transducer portion 30 or open the cutout portion 5k. Therefore, it is possible to further reduce manufacturing costs than in the first embodiment.

In the first embodiment, in order to surely attach the second distal end cover portion 22 to the cutout portion 5k of the first distal end cover portion 21, the distal end cover 1 needs to be formed by a member that is less easily plastically deformed. However, in the present embodiment, the cutout portion 5k is closed by the closing member 65 of the ultrasound transducer portion 30. Therefore, it is unnecessary to limit the member forming the distal end cover 1 to the member that is less easily plastically deformed.

Therefore, since a degree of freedom of a material forming the distal end cover 1 increases, it is possible to adopt a material attaching importance to cost and chemical resistance. Other effects are the same as the effects in the first embodiment explained above.

The ultrasound transducer unit 35 explained above is provided in, for example, the ultrasound endoscope 100. A configuration of an ultrasound endoscope in which the ultrasound transducer unit 35 is provided is explained with reference to FIG. 11.

FIG. 11 is a diagram showing an external appearance of an ultrasound endoscope in which the ultrasound transducer unit shown in FIG. 1 is provided.

A main portion of the ultrasound endoscope 100 includes the elongated insertion portion 110 inserted into a subject, the operation portion 103 provided at the proximal end in the inserting direction S of the insertion portion 110, the universal cord 104 having flexibility extended from the operation portion 103, and the connector 105 provided at an extension end of the universal cord 104.

In the connector 105, a light source connector 105a, an electrical connector 105b, an ultrasound connector 105c, a suction pipe sleeve 105d, and an air/water feeding pipe sleeve 105e are provided.

A light source device that supplies illumination light is detachably attachable to the light source connector 105a. A video processor that performs various kinds of signal processing is detachably attachable to the electrical connector 105b via a signal cable.

An ultrasound observation device is detachably attachable to the ultrasound connector 105c via an ultrasound cable 106 connected to the ultrasound observation device. A suction pump is detachably attachable to the suction pipe sleeve 105d via a suction tube. Further, a water feeding tank is detachably attachable to the air/water feeding pipe sleeve 105e via an air/water feeding tube.

The insertion portion 110 is configured by jointly providing, in order from the distal end side in the inserting direction S, the distal end portion 111, a bending portion 112 formed to be capable of bending in, for example, an up-down direction and a left-right direction, and an elongated flexible tube portion 113 having flexibility.

In the distal end portion 111, the conductor wire portion 31 extended from the ultrasound transducer portion 30 is inserted through to the insertion portion 110, the operation portion 103, the universal cord 104, and the ultrasound connector 105c of the connector 105. In the ultrasound connector 105c, the conductor wire portion 31 is electrically connected to the ultrasound cable 106. As explained above, the conductor wire portion 31 is located while being loosened by the predetermined length T in the insertion portion 110.

The configuration of the ultrasound endoscope 100 shown in FIG. 11 above is only an example. The ultrasound endoscope 100 is not limited to the configuration.

What is claimed is:

1. An ultrasound endoscope comprising:
an objective optical system;
an illumination optical system configured to illuminate a visual field of the objective optical system;
a distal end portion located on a distal end side in an inserting direction of an insertion portion inserted into a subject, at least the objective optical system and the illumination optical system being fixed to the distal end portion;
an ultrasound transducer portion detachably attachable to the distal end portion, at least a region of which including a piezoelectric element projecting further forward in the inserting direction than a distal end face of the distal end portion;
a distal end cover detachably attachable to the distal end portion, the distal end cover covering an outer circumferential surface and the distal end face of the distal end portion such that at least the objective optical system, the illumination optical system, and a region including the piezoelectric element of the ultrasound transducer portion are exposed from the distal end face of the distal end portion;
a through-hole formed in the distal end cover to pierce through the distal end cover along the inserting direction and closed by a part of the ultrasound transducer portion when the ultrasound transducer portion is attached to the distal end portion;
a conductor wire portion, one end of which is electrically connected to the piezoelectric element, the conductor wire portion piercing through the distal end portion in the inserting direction and piercing through the through-hole when the ultrasound transducer portion is detached from the distal end portion; and
a conductor wire portion passing portion formed in the distal end cover and configured to communicate with the through-hole and reach an outer circumference of the distal end cover, the conductor wire portion passing portion forming a region where the conductor wire portion passes from the through-hole to an outside of the distal end cover member when the ultrasound transducer portion is detached from the distal end portion.

2. The ultrasound endoscope according to claim 1, wherein the conductor wire passing portion is a cutout portion that communicates with the through-hole formed in the distal end cover.

3. The ultrasound endoscope according to claim 2, wherein the distal end cover includes:
a first distal end cover portion in which the through-hole and the cutout portion are formed; and
a second distal end cover portion detachably attachable to the cutout portion and configured to close the cutout portion after being attached to the cutout portion.

4. The ultrasound endoscope according to claim 2, wherein the ultrasound transducer portion includes a closing member configured to close the cutout portion.

5. The ultrasound endoscope according to claim 4, wherein the closing member is formed integrally with the ultrasound transducer portion.

6. The ultrasound endoscope according to claim 1, wherein the conductor wire passing portion is a slit, one end of which communicates with the through-hole formed in the distal end cover and the other end of which reaches the outer circumference of the distal end cover.

7. The ultrasound endoscope according to claim 1, wherein
the conductor wire portion is inserted through the insertion portion in a state in which the conductor wire portion is loosened by a predetermined length in the insertion portion, and
when the ultrasound transducer portion is detached from the distal end portion, the ultrasound transducer portion can project further forward than the distal end face of the distal end portion by the length of the loosening of the conductor wire portion in the insertion portion.

8. The ultrasound endoscope according to claim 1, wherein a size of the through-hole is small compared with a largest cross section among cross sections in a surface direction of the ultrasound transducer portion.
